(19) United States
(12) Patent Application Publication
(10) Pub. No.: US 2020/0355151 A1
(43) Pub. Date: Nov. 12, 2020

(54) VEHICLE REMOTE CONTROL SYSTEM AND VEHICLE REMOTE CONTROL METHOD

(71) Applicant: TOYOTA JIDOSHA KABUSHIKI KAISHA, Toyota-shi (JP)

(72) Inventors: Katsutoshi CHAYA, Toyota-shi (JP); Shingo MORI, Miyoshi-shi (JP); Takehiro OKADA, Nisshin-shi (JP)

(73) Assignee: TOYOTA JIDOSHA KABUSHIKI KAISHA, Toyota-shi (JP)

(21) Appl. No.: 16/838,083

(22) Filed: Apr. 2, 2020

(30) Foreign Application Priority Data
May 8, 2019 (JP) 2019-088083

(51) Int. Cl. F02N 11/08; G05D 1/00; B60S 1/02; B60H 1/00; G01N 33/00

(52) U.S. Cl. CPC F02N 11/0807; G05D 1/0016; B60S 1/023; G05D 2201/0213; B60H 1/00735; G01N 33/0004; G05D 1/0027

(57) ABSTRACT

A vehicle remote control system includes a vehicle. The vehicle remote control system includes: a control unit that starts a change of a state of the vehicle to a predetermined state when a predetermined start condition is satisfied; a calculation unit that calculates an estimated required period required to change to reach the predetermined state; and a notification unit that notifies, to a user, estimation information about the calculated estimated required period. The user can be notified of the period required for the state of the vehicle to change to reach the predetermined state.

This nonprovisional application is based on Japanese Patent Application No. 2019-088083 filed on May 8, 2019, with the Japan Patent Office, the entire contents of which are hereby incorporated by reference.

### BACKGROUND

### Field

The present disclosure relates to a vehicle remote control system and a vehicle remote control method, particularly, a vehicle remote control system and a vehicle remote control method, which are suitable to notify estimation information about a vehicle to a user.

### Description of the Background Art

Conventionally, there has been a remote air conditioning starting system that determines whether or not it is necessary to remove frost on a window of a vehicle and that notifies that it is necessary to remove the frost when it is determined so (for example, see Japanese Patent Laying-Open No. 2018-122837).

### SUMMARY

Even though it is notified that it is necessary to remove the frost, the user does not know a period of time required to change to reach a state in which the frost is removed, disadvantageously.

The present disclosure has been made to solve such a problem, and has an object to provide a vehicle remote control system and a vehicle remote control method, by each of which a user can be notified of estimation information about a period required for a state of the vehicle to change to reach a predetermined state.

A vehicle remote control system according to the present disclosure includes a vehicle. The vehicle remote control system includes: a control unit that starts a change of a state of the vehicle to a predetermined state when a predetermined start condition is satisfied; a calculation unit that calculates an estimated required period required to change to reach the predetermined state; and a notification unit that notifies, to a user, estimation information about the estimated required period calculated by the calculation unit.

The predetermined start condition is a condition that a remote instruction is received from the user, and the estimation information includes an estimated time at which the state of the vehicle reaches the predetermined state.

The predetermined start condition is a condition that a time to start the change of the state of the vehicle to the predetermined state is reached, the time to start the change of the state of the vehicle preceding, by the estimated required period, a scheduled departure time set previously, and the estimation information includes a time at which the predetermined start condition is satisfied.

The vehicle includes: a window; and a removal device that removes, by heat, a deposit originating from water and adhered to the window. The predetermined state is a state in which the deposit is removed by the removal device to such a predetermined criterion that a field of view of the user through the window during driving of the vehicle is able to be secured. The control unit controls the removal device to start the change of the state of the vehicle to the predetermined state.

The vehicle includes an air conditioner in a passenger compartment. The predetermined state is a state in which inside of the passenger compartment is air-conditioned to a predetermined temperature by the air conditioner. The control unit controls the air conditioner to start the change of the state of the vehicle to the predetermined state.

The vehicle remote control system further includes a collection unit that collects information, used to calculate the estimated required period, about the state of the vehicle. The calculation unit calculates the estimated required period using the information collected by the collection unit.

The information about the state of the vehicle is weather information used to calculate the estimated required period.

The information about the state of the vehicle is information, used to calculate the estimated required period, about a period of time from start of a change of a state of another vehicle to start of driving of the another vehicle.

The vehicle includes: an internal combustion engine; and a sensor that detects a concentration of a noxious component in an external air, the noxious component being included in exhaust gas from the internal combustion engine. The control unit starts to change the state of the vehicle to the predetermined state by operating the internal combustion engine. When the concentration detected by the sensor is more than or equal to a predetermined value, the control unit stops the internal combustion engine.

According to another aspect of the present disclosure, a vehicle remote control method is a vehicle remote control method in a vehicle remote control system including a vehicle, and includes: starting a change of a state of the vehicle to a predetermined state when a predetermined start condition is satisfied; calculating an estimated required period required to change to reach the predetermined state; and notifying, to a user, estimation information about the calculated estimated required period.

The foregoing and other objects, features, aspects and advantages of the present disclosure will become more apparent from the following detailed description of the present disclosure when taken in conjunction with the accompanying drawings.

### BRIEF DESCRIPTION OF THE DRAWINGS

FIG. 1 shows an overview of a configuration of a vehicle remote control system according to the present embodiment.

FIG. 2 is a flowchart showing a flow of control in a vehicle remote control system according to a first embodiment.

FIG. 3 is a flowchart showing a flow of a possible riding time calculation process in the embodiment.

FIG. 4 is a flowchart showing a flow of control in a vehicle remote control system according to a second embodiment.

FIG. 5 is a flowchart showing a flow of control in a vehicle remote control system according to a third embodiment.

FIG. 6 is a flowchart showing a flow of a starting time calculation process in the third embodiment.

### DESCRIPTION OF THE PREFERRED EMBODIMENTS

The following describes embodiments of the present disclosure with reference to figures in detail. It should be noted that the same or corresponding portions in the figures are given the same reference characters and are not described repeatedly.

<Configuration of System>

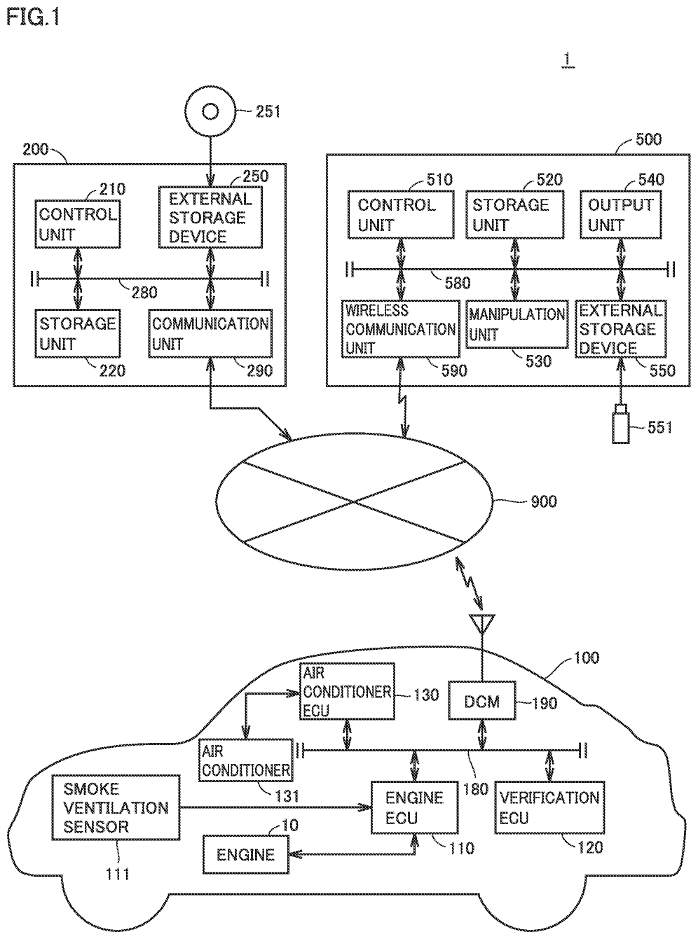

FIG. 1 shows an overview of a configuration of a vehicle remote control system 1 according to the present embodiment. With reference to FIG. 1, vehicle remote control system 1 includes a vehicle 100, a center server 200, and a mobile terminal 500. Vehicle 100, center server 200, and mobile terminal 500 can communicate with one another via a communication network 900. Communication network 900 includes: a private network such as a LAN (Local Area Network) or a VPN (Virtual Private Network); and a public network, such as the Internet, a public network, or a public wireless LAN.

Vehicle 100 includes an engine 10, an engine ECU (Electronic Control Unit) 110, a verification ECU 120, an air conditioner ECU 130, and a DCM (Data Communication Module) 190. Engine ECU 110, verification ECU 120, air conditioner ECU 130, and DCM 190 are connected to one another via an onboard wired network 180 such as a CAN (Controller Area Network), for example.

Engine 10 is an internal combustion engine such as a gasoline engine, and generates driving power of vehicle 100. Engine ECU 110 is a controller that controls engine 10 in accordance with an instruction from a user, and includes: a control unit that controls an operation of engine 10; and a storage unit that stores predetermined information.

The storage unit of engine ECU 110 includes: a RAM (Random Access Memory) used as a workspace required to execute a program by the control unit of engine ECU 110; and a ROM (Read Only Memory) that stores a program to be executed by the control unit. Moreover, program and data for performing a predetermined process are read from the ROM or the like and are stored in the RAM.

The control unit of engine ECU 110 is constituted of an MPU (Micro Processing Unit) and an auxiliary circuit therefor. The control unit performs a predetermined process in accordance with the program and data stored in the storage unit, processes data received from other devices (for example, verification ECU 120, air conditioner ECU 130, DCM 190, and the like) connected to onboard network 180, stores the processed data into the storage unit, and outputs the processed data to another device.

Verification ECU 120 has the same configuration as that of engine ECU 110. Verification ECU 120 is capable of wireless communication with a key held by the user via a transmission/reception antenna, verifies an ID code stored in the key against an ID code stored in verification ECU 120, and permits engine ECU 110 to start engine 10 only when the ID codes coincide with each other. Moreover, the same ID code as the ID code stored in the key held by the user is previously stored in mobile terminal 500 of the user. Verification ECU 120 receives the ID code from mobile terminal 500 of the user via center server 200 and DCM 190, verifies the received ID code against the ID code stored in verification ECU 120, and permits engine ECU 110 to start engine 10 only when the ID codes coincide with each other.

Air conditioner ECU 130 has the same configuration as that of engine ECU 110. Air conditioner ECU 130 is a controller that controls an air conditioner 131 in accordance with an instruction from the user or an instruction from another ECU. Air conditioner 131 is switched to one of a defroster mode, a heating mode, and a cooling mode by air conditioner ECU 130, and is operated in accordance with the switched mode. The defroster mode is a mode in which dehumidified warm air is blown to a windshield and a side window. The heating mode is a mode in which inside of the passenger compartment is heated in accordance with setting temperature and air sending rate adjusted by the user. The cooling mode is a mode in which the inside of the passenger compartment is cooled in accordance with setting temperature and air sending rate adjusted by the user.

Smoke ventilation sensor 111 detects a concentration of a noxious component, such as carbon monoxide (CO), included in external air introduced into the passenger compartment by air conditioner 131, and outputs a detection result to engine ECU 110. When the detection result by smoke ventilation sensor 111 indicates that the concentration of the noxious component is more than or equal to a predetermined value representing a high level thereof and is abnormal, engine ECU 110 stops engine 10.

DCM 190 can communicate with center server 200 via communication network 900, transmits information from verification ECU 120, engine ECU 110, and the like to center server 200, and provides, to verification ECU 120, engine ECU 110, and the like, information received from center server 200.

Center server 200 includes a control unit 210, a storage unit 220, an external storage device 250, and a communication unit 290. Control unit 210, storage unit 220, external storage device 250, and communication unit 290 are connected to one another via a bus 280.

Storage unit 220 includes: a RAM (Random Access Memory) used as a workspace required to execute a program by control unit 210; and a ROM (Read Only Memory) that stores a program to be executed by control unit 210. Moreover, program and data for performing a predetermined process are read from the ROM or the like and are stored in the RAM.

External storage device 250 is constituted of a storage device such as a hard disk drive, a DVD (Digital Versatile Disk) drive, a memory card reader/writer, or the like. External storage device 250 magnetically, optically, or electrically records, onto storage medium 251, predetermined data or program received from control unit 210, and reads predetermined data or program from storage medium 251 and provides it to control unit 210. Examples of storage medium 251 include: a magnetic disk such as a hard disk; an optical disk such as a DVD-ROM (Digital Versatile Disk Read Only Memory); a memory card; a USB (Universal Serial Bus) memory; or the like.

Communication unit 290 transmits and receives data to and from an external device (for example, vehicle 100, mobile terminal 500, or the like) via communication network 900. Communication unit 290 transmits, to outside, data received from a device connected to bus 280 such as control unit 210, and provides externally received data to a device connected to bus 280 such as control unit 210.

Control unit 210 is constituted of an MPU (Micro Processing Unit) and an auxiliary circuit therefor. Control unit 210 controls storage unit 220, communication unit 290, and external storage device 250, performs a predetermined process in accordance with program and data stored in storage unit 220, processes data received from communication unit 290 and external storage device 250, and stores the processed data into storage unit 220, outputs the processed data from communication unit 290 to another device, or stores the processed data into storage medium 251 of external storage device 250.

It should be noted that in the present embodiment, center server 200 includes no manipulation unit and no display unit, but is not limited to this. Center server 200 may include a manipulation unit and a display unit. The manipulation unit may include a keyboard and a mouse, and a manipulate signal indicating a content of a manipulation input in center server 200 by manipulating the keyboard and the mouse of the manipulation unit may be provided to control unit 210. The display unit includes an LCD, and the LCD may present an image corresponding to image data received from control unit 210.

Mobile terminal 500 includes a control unit 510, a storage unit 520, a manipulation unit 530, an output unit 540, an external storage device 550, and a wireless communication unit 590. Control unit 510, storage unit 520, manipulation unit 530, output unit 540, external storage device 550, and wireless communication unit 590 are connected to one another via a bus 580. Control unit 510 and storage unit 520 are the same as control unit 210 of center server 200 and storage unit 220, and are therefore not described repeatedly.

External storage 550 is constituted of a memory card reader/writer. External storage device 550 electrically records, onto a storage medium 551, such as a memory card or a USB (Universal Serial Bus) memory, predetermined data or program received from control unit 510, reads it from storage medium 551, and provides it to control unit 510. It should be noted that external storage device 550 may be constituted of a storage device, such as a hard disk drive, a flexible disk drive, a MO (Magneto-Optical disk) drive, a CD (Compact Disc) drive, or a DVD (Digital Versatile Disk) drive.

Manipulation unit 530 includes a touch panel and a manipulation button for inputting numerals, alphabet, other characters and the like, such as a telephone number and various types of data. It should be noted that manipulation unit 530 may include another portion for manipulation. When manipulation unit 530 is manipulated by the user, a manipulation signal according to the manipulation is sent from manipulation unit 530 to control unit 510. Control unit 510 controls each portion of mobile terminal 500 in accordance with the manipulation signal from manipulation unit 530.

Wireless communication unit 590 is controlled by control unit 510 to receive, via a public network and an antenna, a wireless signal from another mobile terminal 500 or fixed-line telephone with which communication is made, convert the received wireless signal into an audio signal and send the converted audio signal to an audio input/output unit, and convert the audio signal from the audio input/output unit into a wireless signal and transmit, via an antenna and a communication facility of a telecommunications service operator, to another mobile terminal 500 or fixed-line telephone with which communication is made.

Moreover, wireless communication unit 590 is controlled by control unit 510 to receive a wireless signal via the public network and the antenna from a device capable of data communication such as center server 200 or another mobile terminal 500, convert the received wireless signal into data and store the converted data into storage unit 520 or send the converted data to output unit 540 in order to display the data, and convert data to be transmitted into a wireless signal and transmit, via an antenna and a communication facility of a telecommunications service operator, to center server 200 or another mobile terminal 500 with which data communication is made.

Moreover, wireless communication unit 590 is controlled by control unit 510 to exchange data with other devices capable of network communication via a public wireless LAN or private network wireless LAN, such as center server 200 and other mobile terminals 500.

Output unit 540 includes a display and a speaker. Output unit 540 is controlled by control unit 510 to present an image signal on the display as an image and outputs an audio signal from the speaker as a sound. The image signal and audio signal are obtained by converting, by control unit 510, information received by wireless communication unit 590, information stored in storage unit 520, or information read from storage medium 551 in external storage device 550.

### First Embodiment

Conventionally, there has been a remote air conditioning starting system that determines whether or not it is necessary to remove frost on a window of vehicle 100 and that notifies that it is necessary to remove the frost when it is determined so. Even though it is notified that it is necessary to remove the frost, the user does not know a period of time required to change to reach a state in which the frost is removed, disadvantageously.

To address this, vehicle remote control system 1 according to the present disclosure includes vehicle 100, starts a change of a state of vehicle 100 to a predetermined state when a predetermined start condition is satisfied, calculates an estimated required period required to change to reach the predetermined state, and notifies, to the user, estimation information about the calculated estimated required period. Accordingly, the user can be informed of the estimation information about the period required for the state of vehicle 100 to change to reach the predetermined state.

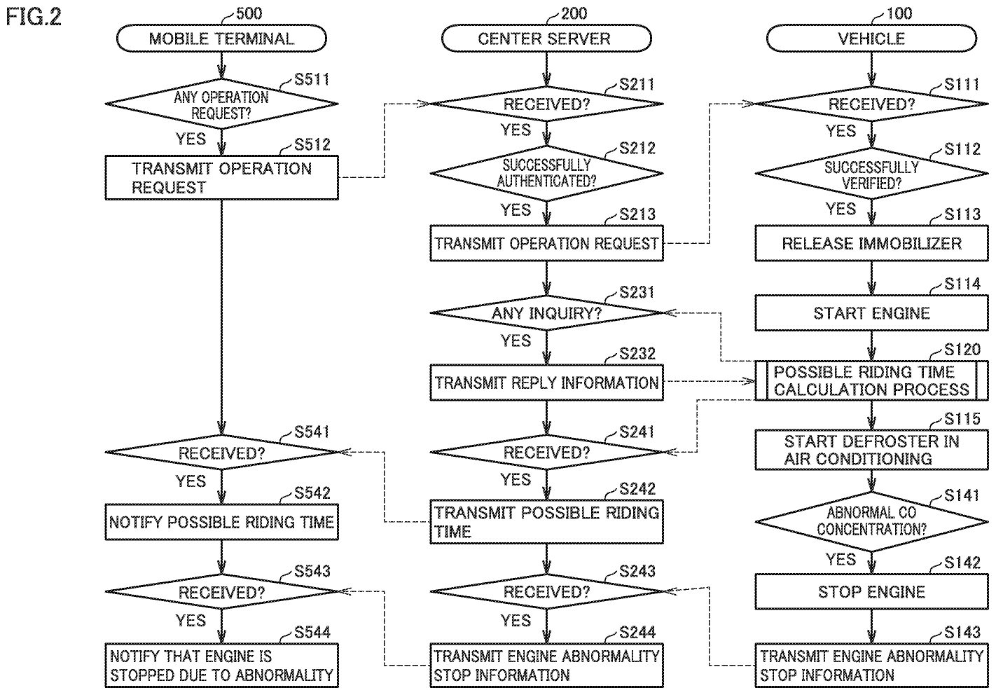

The following describes vehicle remote control system 1 according to the present embodiment. FIG. 2 is a flowchart showing a flow of control in vehicle remote control system 1 according to the first embodiment. In a season during which an external air temperature is low, a deposit originating from water, such as ice frost or snow, is adhered onto the windshield of vehicle 100, or condensation occurs inside or outside the windshield, with the result that a field of view for driving of vehicle 100 by the user may be unable to be secured. Moreover, a temperature in the passenger compartment of vehicle 100 becomes extremely low to adversely affect the user in driving vehicle 100. Vehicle remote control system 1 can operate vehicle 100 by way of remote control with mobile terminal 500 in order to remove a deposit on a window or attain an appropriate temperature in the passenger compartment before the user rides on vehicle 100.

With reference to FIG. 2, in mobile terminal 500 of the user, control unit 510 determines whether or not an operation request for removing a deposit on a window of vehicle 100 or attaining an appropriate temperature in the passenger compartment is input by the user manipulating manipulation unit 530 (step S511). When it is determined that no operation request is input, control unit 510 performs another process. When it is determined that the operation request is input (YES in step S511), control unit 510 transmits, from wireless communication unit 590 to center server 200, an operation request including an ID code of a key of vehicle 100 stored previously in storage unit 520 (step S512).

In center server 200, control unit 210 determines whether or not the operation request from mobile terminal 500 is received at communication unit 290 (step S211). When it is determined that no operation request is received, control unit 510 performs another process. When it is determined that the operation request is received (YES in step S211), control unit 510 performs authentication as to whether or not the ID code included in the received operation request is a valid ID code of a key held by the user of previously registered vehicle 100 (step S212). When it is determined that the ID code included in the operation request is not valid as a result of the authentication, a process in the case where the ID code is not valid is performed (for example, a process for transmitting, to mobile terminal 500, information indicating that the ID code is not valid).

When it is determined that the ID code included in the operation request is valid as a result of the authentication (YES in step S212), control unit 210 transmits the operation request including the ID code to previously registered vehicle 100 of the user of mobile terminal 500 (step S213).

Verification ECU 120 of vehicle 100 determines whether or not the operation request is received from center server 200 via DCM 190 (step S111). When it is determined that no operation request is received, verification ECU 120 performs another process. When it is determined that the operation request is received (YES in step S111), verification ECU 120 verifies the ID code included in the received operation request against the ID code stored previously in the storage unit of verification ECU 120 (step S112). When it is determined that the ID codes do not coincide with each other as a result of verification, verification ECU 120 performs a process in the case where the ID codes do not coincide with each other (for example, a process for transmitting, to mobile terminal 500 via center server 200, information indicating that the ID code does not coincide with the ID code of vehicle 100). When it is determined that the ID codes coincide with each other as a result of the verification (YES in step S112), verification ECU 120 releases engine 10 from a state in which engine 10 is unable to be started by an immobilizer, thereby bringing into a state in which engine 10 can be started (step S113).

Accordingly, engine 10 can be started, and engine ECU 110 starts engine 10 in accordance with the operation request from center server 200 (step S114).

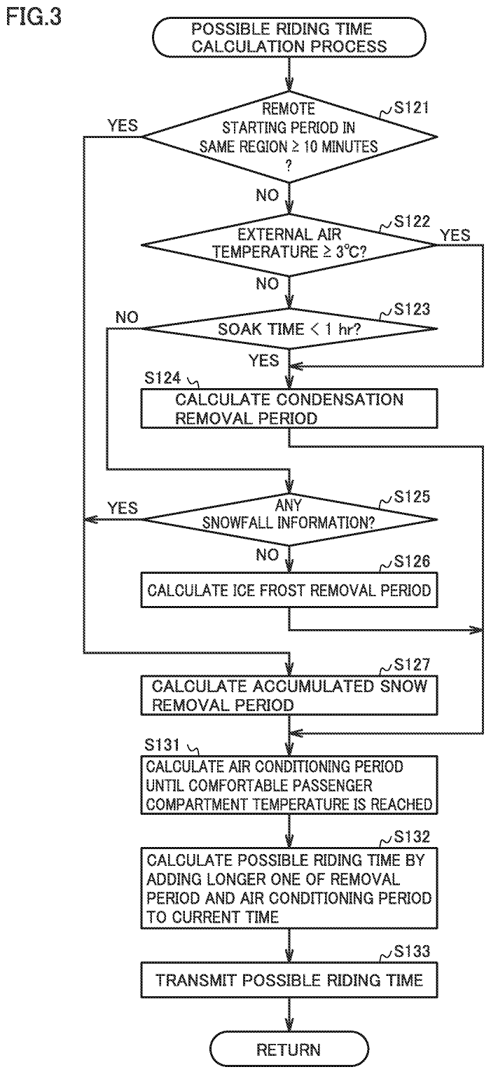

Then, engine ECU 110 performs a possible riding time calculation process (step S120). FIG. 3 is a flowchart showing a flow of the possible riding time calculation process in the present embodiment. Regarding remote starting for starting engines 10 of a plurality of managed vehicles 100 by way of remote control, in center server 200, control unit 210 collects, from the plurality of vehicles 100, respective remote starting periods from starts of engines 10 to starts of driving of vehicles 100. The remote starting periods are stored in storage unit 220.

With reference to FIG. 3, engine ECU 110 of vehicle 100 obtains, from center server 200, an average value of remote starting periods of other vehicles 100 within a predetermined period (for example, a period from 2 or 3 hours before the current time to the current time) in a region in which vehicle 100 is parked (for example, a surrounding region with a radius of predetermined km, the same city, the same town or the same village), and determines whether or not the obtained average value of the remote starting periods is more than or equal to a predetermined period (here, 10 minutes) (step S121).

Turning back to FIG. 2, in order to obtain desired information from center server 200, engine ECU 110 of vehicle 100 makes an inquiry to center server 200 about obtaining of the desired information. Control unit 210 of center server 200 determines whether or not there is an inquiry from vehicle 100 about obtaining of information (step S231). When it is determined that there is no inquiry, control unit 210 performs another process. When it is determined that there is an inquiry (YES in step S231), control unit 210 searches for the desired information to be obtained by vehicle 100, and transmits, to vehicle 100, reply information including the searched desired information (step S232). By receiving the reply information from center server 200, engine ECU 110 of vehicle 100 can obtain the desired information included in the reply information.

With reference to FIG. 3, when it is determined that the remote starting period is not more than or equal to the predetermined period (NO in step S121), engine ECU 110 obtains an external air temperature from an external air temperature sensor, and determines whether or not the obtained external air temperature is more than or equal to a predetermined temperature (here, 3° C.) (step S122). When it is determined that the external air temperature is not more than or equal to the predetermined temperature (NO in step S122), engine ECU 110 determines whether or not a soak time is less than a predetermined period (for example, 1 hour) (step S123). The soak time is a period of time during which engine 10 has not been operated before engine 10 is started.

When it is determined that the external air temperature is more than or equal to the predetermined temperature (YES in step S122) or when it is determined that the soak time is less than the predetermined period (YES in step S123), engine ECU 110 determines whether or not condensation occurs inside or outside a window such as the windshield in accordance with temperature and humidity in the passenger compartment, temperature and humidity outside the vehicle, a temperature difference between inside and outside of the passenger compartment, the soak time, and the like, and calculates a condensation removal period when it is determined that condensation occurs (step S124). When it is determined that no condensation occurs, the condensation removal period is set to 0.

The determination for occurrence of condensation and the calculation of the condensation removal period can be performed, for example, as follows. In an actual vehicle experiment with vehicle 100, whether or not condensation occurs inside or outside a window is examined with respect to each of combinations of temperatures and humidities inside the passenger compartment and outside the vehicle and soak times by variously changing the combinations of temperatures and humidities inside the passenger compartment and outside the vehicle and soak times. Results of the examination are stored as a table in advance in the storage unit of engine ECU 110. Accordingly, temperatures and humidities inside the passenger compartment and outside the vehicle are detected by sensors, a soak time is specified, and occurrence of condensation can be estimated based on the table stored in the storage unit as well as the results of detection of the sensors and the soak time.

Moreover, for each of conditions of the combinations of the temperatures and humidities inside the passenger compartment and outside the vehicle and the soak times, a period required to remove the condensation inside and outside the window is examined by operating air conditioner 131 in the defroster mode under the strongest conditions (highest air sending rate and highest air sending temperature). Results of the examination are stored as a table in advance in the storage unit of engine ECU 110. Accordingly, temperatures and humidities inside the passenger compartment and outside the vehicle are detected by sensors, a soak time is specified, and a condensation removal period can be estimated based on the table stored in the storage unit as well as the results of detection of the sensors and the soak time.

Moreover, occurrence of condensation and a condensation removal period may be estimated in the following manner: by way of an actual vehicle experiment and a simulation experiment, a correlation function for occurrence of condensation and a condensation removal period with respect to various temperatures and humidities inside the passenger compartment and outside the vehicle and various soak times is prepared previously, and values of the temperatures and humidities inside the passenger compartment and outside the vehicle as detected by sensors and the soak time are substituted into the correlation function.

Moreover, the condensation removal period can be estimated in the following manner: an amount of heat required to evaporate condensed water is determined based on the area of the window and a temperature (approximated by an external air temperature) of the window before heating, and is divided by an amount of heat (experimental value) per unit period that can be applied to the window by sending air from air conditioner 131 in the defroster mode.

When it is determined that the soak time is not less than a predetermined period (NO in step S123), engine ECU 110 obtains, from center server 200, snowfall information for a region in which vehicle 100 is parked (for example, a surrounding region with a radius of predetermined km, the same city, the same town or the same village), and determines whether or not there is snowfall information indicating an accumulated snow depth of more than or equal to predetermined cm (for example, 2 or 3 cm) (step S125). It should be noted that control unit 210 of center server 200 regularly (for example, every one hour) obtains snowfall information for every region of the country from a weather information site or the like, and stores the snowfall information in storage unit 220.

When it is determined that there is no snowfall information indicating an accumulated snow depth of more than or equal to the predetermined cm (NO in step S125), engine ECU 110 determines whether or not ice frost is adhered to a window such as the windshield. When it is determined that ice frost is adhered thereto, an ice frost removal period is calculated (step S126). When it is determined that ice frost is not adhered, the ice frost removal period is set to 0.

The determination for adhesion of ice frost and the calculation of the ice frost removal period can be performed as follows, for example. In an actual vehicle experiment with vehicle 100, whether or not ice frost is adhered to outside of the window is examined with respect to each of combinations of temperatures and humidities outside the vehicle and soak times by variously changing the temperature and humidity outside the vehicle and the soak time. Results of the examination are stored as a table in advance in the storage unit of engine ECU 110. Accordingly, temperature and humidity outside the vehicle are detected by sensors, a soak time is specified, and adhesion of ice frost can be estimated based on the table stored in the storage unit as well as the results of detection of the sensors and the soak time.

Moreover, for each of conditions of the combinations of the temperatures and humidities outside the vehicle and the soak times, a period of time required to remove the ice frost outside the window is examined by operating air conditioner 131 in the defroster mode under the strongest conditions (highest air sending rate and highest air sending temperature). Results of the examination are stored as a table in advance in the storage unit of engine ECU 110. Accordingly, temperature and humidity outside the vehicle are detected by sensors, a soak time is specified, and an ice frost removal period can be estimated based on the table stored in the storage unit as well as the results of detection of the sensors and the soak time.

Moreover, the adhesion of ice frost and the ice frost removal period may be estimated in the following manner: by way of an actual vehicle experiment and a simulation experiment, a correlation function for the adhesion of ice frost and the ice frost removal period with respect to various temperatures and humidities outside the vehicle and various soak times is prepared previously, and values of the temperature and humidity outside the vehicle as detected by sensors and the soak time are substituted into the correlation function.

Moreover, the ice frost removal period can be estimated in the following manner: an amount of heat required to melt the adhered ice frost is determined based on the area of the window and a temperature (approximated by an external air temperature) of the window before heating, and is divided by an amount of heat (experimental value) per unit period that can be applied to the window by sending air from air conditioner 131 in the defroster mode.

When it is determined that the remote starting period is more than or equal to the predetermined period (YES in step S121) or when it is determined that there is snowfall information indicating an accumulated snow depth of more than or equal to predetermined cm (YES in step S125), it is determined whether or not snow is accumulated on a window such as the windshield. When it is determined that snow is accumulated thereon, an accumulated snow removal period is calculated (step S127). When it is determined that snow is not accumulated, an accumulated snow removal period is set to 0. It should be noted that when part of the accumulated snow in contact with the window is melted, the other part of the accumulated snow can be removed by scraping off the other part of the accumulated snow by force of human or force of a wiper. Therefore, the accumulated snow removal period is not a period of time until all the accumulated snow is melted, but is a period of time until the part of the accumulated snow in contact with the window is melted.

The determination for accumulation of snow and the calculation of the accumulated snow removal period can be performed as follows, for example. In an actual vehicle experiment with vehicle 100, various amounts of snow are artificially fallen. An accumulated snow depth to the window is examined with respect to each of combinations of amounts of fallen snow, temperatures outside the vehicle, and soak times by variously changing the temperature outside the vehicle and the soak time. Results of the examination are stored as a table in advance in the storage unit of engine ECU 110. Accordingly, an amount of fallen snow is obtained from center server 200, a temperature outside the vehicle is detected by a sensor, a soak time is specified, and an accumulated snow depth can be estimated based on the table stored in the storage unit, the amount of fallen snow obtained from center server 200, the detection result of the sensor, and the soak time.

Moreover, for each of conditions of the combinations of the amounts of fallen snow, the temperatures outside the vehicle and the soak times, a period required to remove the snow accumulated on the window is examined by operating air conditioner 131 in the defroster mode under the strongest conditions (highest air sending rate and highest air sending temperature). Results of the examination are stored as a table in advance in the storage unit of engine ECU 110. Accordingly, an amount of fallen snow is obtained from center server 200, a temperature outside the vehicle is detected by a sensor, a soak time is specified, and an accumulated snow removal period can be estimated based on the table stored in the storage unit, the amount of fallen snow obtained from center server 200, the detection result of the sensor, and the soak time.

Moreover, the accumulated snow depth and the accumulated snow removal period may be estimated in the following manner: by way of an actual vehicle experiment and a simulation experiment, a correlation function for an accumulated snow depth and an accumulated snow removal period with respect to various amounts of fallen snow, various temperatures outside the vehicle and various soak times is prepared previously, and an amount of fallen snow obtained from center server 200, a value of temperature outside the vehicle as detected by the sensor, and a soak time are substituted into the correlation function.

Moreover, the accumulated snow removal period can be estimated in the following manner: an amount of heat required to melt part of the accumulated snow in contact with the window is determined based on the area of the window and a temperature (approximated by an external air temperature) of the window before heating, and is divided by an amount of heat (experimental value) per unit period that can be applied to the window by sending air from air conditioner 131 in the defroster mode.

After step S124, step S126, and step S127, engine ECU 110 calculates an air conditioning period until a passenger compartment temperature set previously by the user is reached (step S131).

The calculation of the air conditioning period can be performed as follows, for example. In an actual vehicle experiment with vehicle 100, by variously changing the temperature outside the vehicle, increase rate and decrease rate of the passenger compartment temperature when operating air conditioner 131 in the heating mode and the cooling mode under the strongest conditions (highest air sending rate and highest air sending temperature) are examined with respect to each of the temperatures outside the vehicle. Results of the examination are stored as a table in advance in the storage unit of engine ECU 110. Accordingly, the temperature outside the vehicle is detected by the sensor, and the increase rate or decrease rate of the passenger compartment temperature can be estimated based on the table stored in the storage unit and the detection result of the sensor. Then, by dividing, by the increasing rate or the decreasing rate, a difference between a target passenger compartment temperature and a passenger compartment temperature detected by the sensor, the air conditioning period can be estimated.

Moreover, the increasing rate of the passenger compartment temperature may be estimated in the following manner: by way of an actual vehicle experiment and a simulation experiment, a correlation function for the increasing rate of the passenger compartment temperature with respect to various temperatures outside the vehicle is prepared previously, and a value of the temperature outside the vehicle as detected by the sensor is substituted into the correlation function.

Next, engine ECU 110 calculates a possible riding time obtained by adding, to a current time, a longer one of the removal period calculated in step S124, step S126, or step S127 and the air conditioning period calculated in step S131 (step S132), transmits the calculated possible riding time to center server 200 (step S133), and returns the execution process to the process of FIG. 2 from which this possible riding time calculation process is invoked.

Turning back to FIG. 2, after the possible riding time calculation process, air conditioner ECU 130 starts air conditioner 131 in accordance with the determination result in the possible riding time calculation process (step S115). Specifically, when it is determined that condensation occurs, ice frost is adhered, or snow is accumulated in the possible riding time calculation process, air conditioner 131 is started in the defroster mode. Moreover, when it is determined that condensation does not occur, ice frost is not adhered, or snow is not accumulated, air conditioner 131 is started in the heating mode if the passenger compartment temperature set previously by the user is higher than the current passenger compartment temperature, or air conditioner 131 is started in the cooling mode if the passenger compartment temperature set previously by the user is lower than the current passenger compartment temperature.

Control unit 210 of center server 200 determines whether or not the possible riding time is received from vehicle 100 (step S241). When it is determined that the possible riding time is not received, control unit 210 performs another process. When it is determined that the possible riding time is received (YES in step S241), control unit 210 transmits the received possible riding time to mobile terminal 500 of the user of vehicle 100 (step S242).

Control unit 510 of mobile terminal 500 determines whether or not the possible riding time is received from center server 200 (step S541). When it is determined that the possible riding time is not received, control unit 510 performs another process. When it is determined that the possible riding time is received (YES in step S541), control unit 510 notifies the received possible riding time by way of output from the display or speaker of output unit 540 (step S542). Accordingly, the user can be informed of a time at which the user can ride on and drive vehicle 100.

After engine 10 is started by way of remote control, engine ECU 110 of vehicle 100 determines whether or not the concentration of a noxious component (for example, CO) of external air detected by smoke ventilation sensor 111 is more than or equal to a predetermined value representing a high level thereof and is abnormal (step S141). When it is determined that the concentration of the noxious component is not abnormal, engine ECU 110 performs another process.

When it is determined that the concentration of the noxious component is abnormal (YES in step S141), engine ECU 110 stops engine 10 (step S142), and transmits engine abnormality stop information to center server 200 (step S143).

Control unit 210 of center server 200 determines whether or not engine abnormality stop information is received from vehicle 100 (step S243). When it is determined that engine abnormality stop information is not received, control unit 210 performs another process. When it is determined that engine abnormality stop information is received (YES in step S243), control unit 210 transmits the engine abnormality stop information to mobile terminal 500 of the user of vehicle 100 (step S244).

Control unit 510 of mobile terminal 500 determines whether or not the engine abnormality stop information is received from center server 200 (step S543). When it is determined that the engine abnormality stop information is not received, control unit 510 performs another process. When it is determined that the engine abnormality stop information is received (YES in step S543), control unit 510 notifies, by way of an output from the display or speaker of output unit 540, that engine 10 is stopped due to an abnormality (step S544). Accordingly, the user can be informed that engine 10 of vehicle 100 is stopped due to the abnormality, and can be urged to go to the location at which vehicle 100 is parked and check a situation thereof.

### Second Embodiment

In the first embodiment, the possible riding time is calculated in vehicle 100. In a second embodiment, the possible riding time is calculated in center server 200.

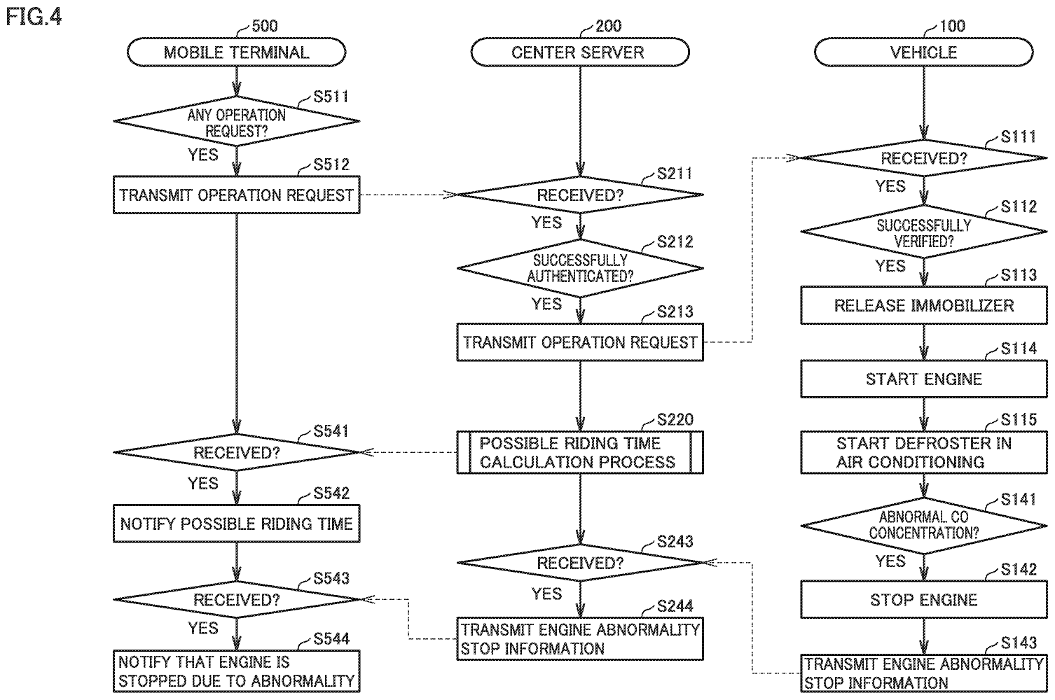

FIG. 4 is a flowchart showing a flow of control in a vehicle remote control system according to the second embodiment. In FIG. 2 of the first embodiment, as shown in step S120, step S231, step S232, and step S241, vehicle 100 makes an inquiry about desired information to center server 200, obtains the desired information, calculates a possible riding time, and transmits the calculated possible riding time to center server 200. With reference to FIG. 4, in the second embodiment, the process for calculating the possible riding time is not performed in vehicle 100, and control unit 210 of center server 200 performs the possible riding time calculation process as shown in FIG. 3 (step S220). In the first embodiment, as shown in step S133 of FIG. 3, the possible riding time is transmitted to center server 200; however, in the second embodiment, as shown in step S220 of FIG. 4, the possible riding time is transmitted to mobile terminal 500.

It should be noted that in the first embodiment, in the possible riding time calculation process, an inquiry about information required for the calculation of the possible riding time is made to center server 200 and the information is obtained therefrom; however, since center server 200 performs the possible riding time calculation process in the second embodiment, center server 200 obtains information required for the calculation of the possible riding time by itself.

### Third Embodiment

In the first embodiment, the possible riding time is calculated in vehicle 100, whereas in the second embodiment, the possible riding time is calculated in center server 200. That is, the possible riding time after the current time at which the user makes the operation request is calculated. In a third embodiment, an scheduled departure time to head for a company or trip is set previously in mobile terminal 500 of the user of vehicle 100, and a time to start engine 10 of vehicle 100 is calculated in order to allow the user to ride on vehicle 100 and depart at the scheduled departure time.

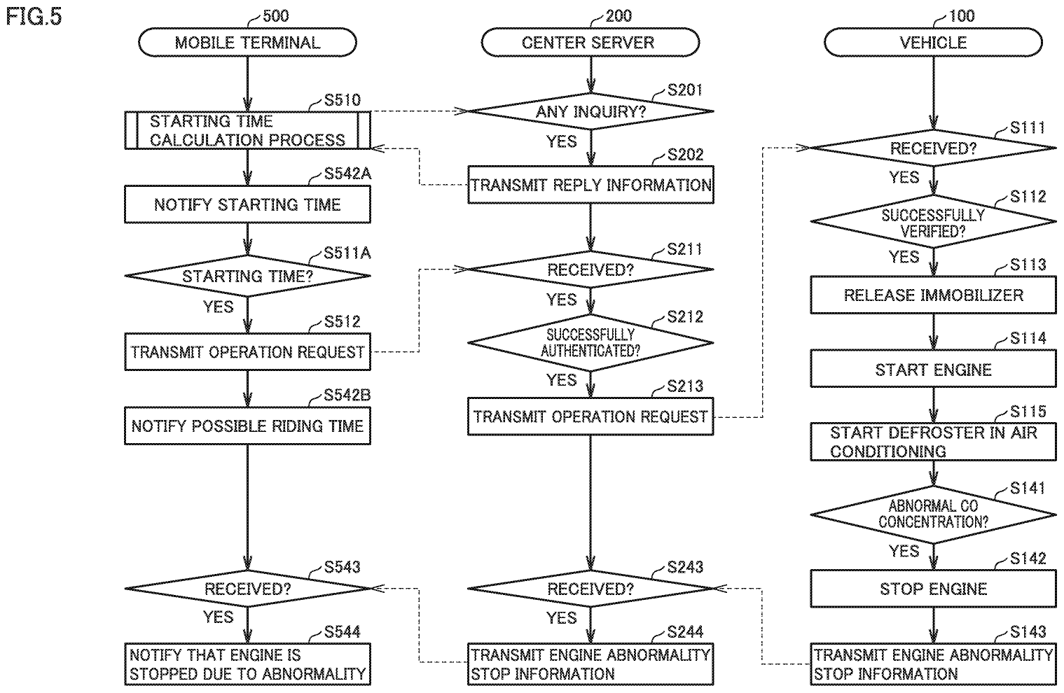

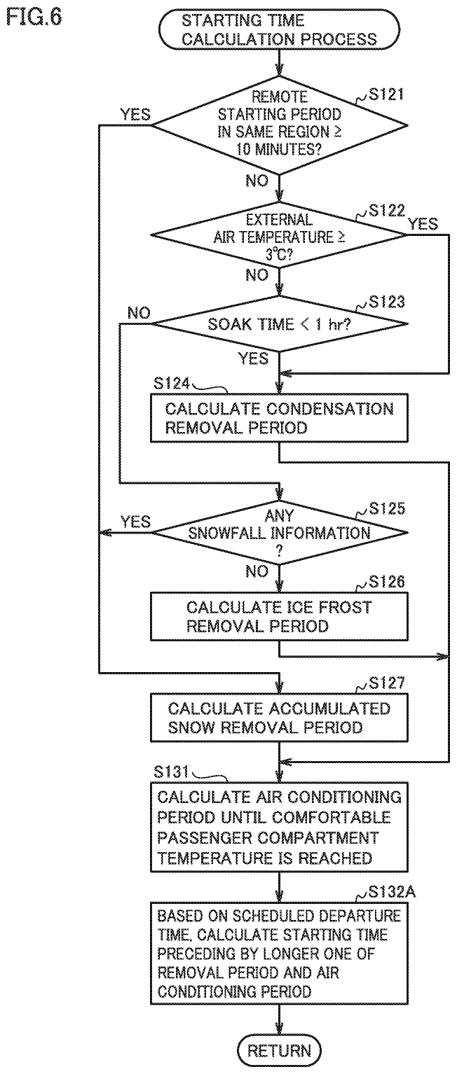

FIG. 5 is a flowchart showing a flow of control in vehicle remote control system 1 according to the third embodiment. With reference to FIG. 5, in mobile terminal 500 of the user, control unit 510 performs a starting time calculation process shown in FIG. 6 at a time preceding, by a predetermined period, a scheduled departure time set previously by the user (step S510). This predetermined period is a period of time with a margin for a period expected to be required to remove a deposit on a window or attain an appropriate temperature in the passenger compartment. For example, the predetermined period is 2 or 3 hours.

FIG. 6 is a flowchart showing a flow of the starting time calculation process in the third embodiment. With reference to FIG. 6, since steps S121 to S131 of the starting time calculation process are the same as steps S121 to S131 of the possible riding time calculation process described with reference to FIG. 3, the same description is not repeated.

Turning back to FIG. 5, as with step S231 and step S232 of FIG. 2, in order to obtain desired information from center server 200 in the starting time calculation process, control unit 510 of mobile terminal 500 makes an inquiry to center server 200 about obtaining of the desired information. Control unit 210 of center server 200 determines whether or not there is an inquiry from mobile terminal 500 about obtaining of information (step S201). When it is determined that there is no inquiry, control unit 210 performs another process. When it is determined that there is an inquiry (YES in step S201), control unit 210 searches for the desired information to be obtained by mobile terminal 500, and transmits, to mobile terminal 500, reply information including the searched desired information (step S202). By receiving the reply information from center server 200, control unit 510 of mobile terminal 500 can obtain the desired information included in the reply information.

With reference to FIG. 6, based on the scheduled departure time set previously by the user, control unit 510 calculates a starting time preceding by a longer one of the removal period calculated in step S124, step S126, or step S127 and the air conditioning period calculated in step S131 (step S132A), and returns the execution process to the process of FIG. 5 from which this starting time calculation process is invoked.

With reference to FIG. 5 again, control unit 510 notifies the estimated starting time calculated by the starting time calculation process, by way of an output from the display or speaker of output unit 540 (step S542A). Accordingly, the user can be informed of the estimated starting time of engine 10 and air conditioner 131 of vehicle 100.

Next, control unit 510 determines whether or not a current time reaches the starting time calculated in step S510 (step S511A). When it is determined that the starting time is not reached, control unit 510 performs another process. When it is determined that the starting time is reached (YES in step S511A), the above-described process of step S512 in FIG. 2 is performed. Accordingly, engine 10 of vehicle 100 is started and air conditioner 131 is started.

Next, control unit 510 notifies the possible riding time (=scheduled departure time) by way of an output from the display or speaker of output unit 540 (step S542B). Accordingly, the user can be informed of a time at which the user can ride on and drive vehicle 100. It should be noted that the possible riding time is the same as the scheduled departure time set previously by the user, and therefore may not be notified. Moreover, the start of engine 10 may be notified.

Since a starting time about the estimated required period required to change to reach a state in which the user can ride on vehicle 100 is thus calculated in mobile terminal 500, the possible riding time about the estimated required period required to change to reach the state in which the user can ride on vehicle 100 does not need to be calculated in vehicle 100 and center server 200 as shown in FIG. 2 of the first embodiment and FIG. 4 of the second embodiment. The other processes of FIG. 5 are the same as those in FIG. 2 of the first embodiment and FIG. 4 of the second embodiment, and therefore the same description is not repeated.

<Modifications>

(1) In the above-described embodiments, condensation, ice frost, and accumulated snow on the window are removed by operating air conditioner 131 in the defroster mode in which dehumidified warm air is blown to the windshield and side window. However, it is not limited to air conditioner 131 as long as it is a removal device that removes, by heat, a deposit originating from water and adhered to the window. For example, the condensation, ice frost, and accumulated snow on the window may be removed by providing electric power to a heating wire embedded in a glass of the window so as to generate heat.

(2) In the above-described embodiments, vehicle 100 includes engine 10. It is not limited to this. Vehicle 100 may be a hybrid vehicle including a motor generator in addition to the engine, may be an electric vehicle including no engine and including a motor generator, or may be a fuel cell vehicle.

(3) In the above-described embodiments, information, such as an instruction, from mobile terminal 500 to vehicle 100 is transmitted via center server 200. However, it is not limited to this. The information, such as an instruction, from mobile terminal 500 to vehicle 100 may be directly transmitted to vehicle 100 not via center server 200.

Moreover, in the above-described embodiments, information, such as an instruction, from mobile terminal 500 to center server 200 is directly transmitted to center server 200. However, it is not limited to this. The information, such as an instruction, from mobile terminal 500 to center server 200 may be transmitted via vehicle 100.

(4) In the above-described embodiments, the check of the ID code is performed not only in vehicle 100 but also in center server 200 as shown in step S112 and step S212 of FIG. 2. However, it is not limited to this. The check may not be performed in center server 200 and may be performed only in vehicle 100.

Moreover, in the above-described embodiments, when an operation request is transmitted from mobile terminal 500 to vehicle 100 via center server 200, engine 10 and air conditioner 131 are started. However, it is not limited to this. Engine 10 and air conditioner 131 may be started also when an operation request is directly transmitted from mobile terminal 500 to vehicle 100.

(5) The above-described embodiments can be recognized as being a disclosure of vehicle remote control system 1 shown in FIG. 1. Alternatively, the above-described embodiments can be recognized as being a disclosure of vehicle 100, center server 200 or mobile terminal 500 included in vehicle remote control system 1. Alternatively, the above-described embodiments can be recognized as being a disclosure of the vehicle remote control method performed in vehicle remote control system 1 or a disclosure of the vehicle remote control program executed in vehicle remote control system 1. Alternatively, the above-described embodiments can be recognized as being a disclosure of the vehicle remote control method performed in vehicle 100, center server 200, or mobile terminal 500 or a disclosure of the vehicle remote control program executed in vehicle 100, center server 200, or mobile terminal 500.

<Configurations and Effects Shown in Above-Described Embodiments>

(1) As shown in FIG. 1, a vehicle remote control system 1 includes a vehicle 100. As shown in FIG. 2 to FIG. 6, vehicle remote control system 1 includes: a control unit (for example, air conditioner ECU 130; step S115 in FIG. 2, FIG. 4 and FIG. 5) that starts a change of a state of vehicle 100 to a predetermined state (for example, a state that a deposit originating from water and adhered to a window is removed, or a state that an temperature in a passenger compartment becomes a predetermined temperature) when a predetermined start condition (for example, a condition that an operation request is made by a user as shown in step S511 of FIG. 2 and FIG. 4, or a condition that a starting time is reached as shown in step S511A of FIG. 6) is satisfied; a calculation unit (for example, engine ECU 110; step S124, step S126, step S127, and step S131 in FIG. 3 and FIG. 6) that calculates an estimated required period (for example, the removal period or air conditioning period in FIG. 3 and FIG. 6) required to change to reach the predetermined state; and a notification unit (for example, output unit 540 of mobile terminal 500; step S542 of FIG. 2 and FIG. 4; step S542A of FIG. 5) that notifies, to a user, estimation information (for example, the possible riding time in FIG. 3, or the remote starting time for engine 10 in FIG. 6) about the estimated required period calculated.

Accordingly, the user can be informed of the estimation information about the period required for the state of vehicle 100 to change to reach the predetermined state.

(2) As shown in FIG. 2 and FIG. 4, the predetermined start condition is a condition (for example, a condition that an operation request is made by the user as shown in step S511 of FIG. 2 and FIG. 4) that a remote instruction is received from the user, and the estimation information includes an estimated time (for example, the possible riding time as shown in step S542 of FIG. 2 and FIG. 4) at which the state of vehicle 100 reaches the predetermined state. Accordingly, the user can be informed of the estimated time for the state of vehicle 100 to reach the predetermined state.

(3) As shown in FIG. 6, the predetermined start condition is a condition (for example, a condition that a starting time is reached as shown in step S511A of FIG. 6) that a time to start the change of the state of vehicle 100 to the predetermined state is reached, the time to start the change of the state of vehicle 100 preceding, by the estimated required period, a scheduled departure time set previously, and the estimation information includes a time (for example, the starting time shown in step S542A of FIG. 5) at which the predetermined start condition is satisfied. Accordingly, the user can be informed of the time to start the change of the state of vehicle 100 to the predetermined state, the time to start the change preceding the scheduled departure time set previously by the estimated required period.

(4) As shown in FIG. 1, vehicle 100 includes: a window; and a removal device (for example, air conditioner 131 that operates in the defroster mode) that removes, by heat, a deposit originating from water and adhered to the window. The predetermined state is a state in which the deposit is removed by the removal device to such a predetermined criterion (for example, a state in which condensation or ice frost is removed, or a state in which part of accumulated snow in contact with the window is melted) that a field of view of the user through the window during driving of vehicle 100 is able to be secured. The control unit (for example, air conditioner ECU 130 of vehicle 100) controls the removal device to start the change of the state of vehicle 100 to the predetermined state (for example, step S115 in FIG. 2, FIG. 4 and FIG. 5). Accordingly, the user can be informed of the estimation information about the period required for the state of vehicle 100 to change to reach a state in which the deposit is removed by the removal device to such a predetermined criterion that the field of view of the user through the window during driving of vehicle 100 can be secured.

(5) As shown in FIG. 1, vehicle 100 includes an air conditioner 131 in a passenger compartment. The predetermined state is a state in which inside of the passenger compartment is air-conditioned by air conditioner 131 to a predetermined temperature set by the user. The control unit (for example, air conditioner ECU 130 of vehicle 100) controls air conditioner 131 to start the change of the state of vehicle 100 to the predetermined state (for example, step S115 of FIG. 2, FIG. 4 and FIG. 5). Accordingly, the user can be informed of the estimation information about the period required for the state of vehicle 100 to change to reach the state in which the inside of the passenger compartment is air-conditioned by air conditioner 131 to the predetermined temperature set by the user.

(6) Vehicle remote control system 1 further includes a collection unit (for example, engine ECU 110 of vehicle 100; step S121 and step S125 in FIG. 3 and FIG. 6) that collects information (for example, remote starting periods of a plurality of vehicle 100; snowfall information), used to calculate the estimated required period, about the state of vehicle 100. The calculation unit calculates the estimated required period using the information collected by the collection unit (for example, engine ECU 110; step S124, step S126, step S127, and step S131 in FIG. 3 and FIG. 6). Accordingly, the estimated required period can be calculated more correctly.

(7) The information about the state of vehicle 100 is weather information (for example, snowfall information) used to calculate the estimated required period.

(8) The information about the state of vehicle 100 is information, used to calculate the estimated required period, about a period of time (for example, remote starting period) from start of a change of a state of another vehicle 100 to start of driving of the another vehicle 100.

(9) Vehicle 100 includes: an internal combustion engine (for example, engine 10); and a sensor (for example, smoke ventilation sensor 111) that detects a concentration of a noxious component (for example, carbon monoxide) in an external air, the noxious component being included in exhaust gas from the internal combustion engine. The control unit starts to change the state of the vehicle to the predetermined state by operating the internal combustion engine. When the concentration detected by the sensor is more than or equal to a predetermined value, the control unit stops the internal combustion engine (for example, engine ECU 110; step S141 and step S142 in FIG. 2, FIG. 4 and FIG. 5). Accordingly, when the internal combustion engine is remotely started, even if the vehicle is parked at a location with poor ventilation such as a garage and a person is in the passenger compartment or is in the vicinity of the vehicle such as inside of the garage, the internal combustion engine is stopped before the concentration of the noxious component in air within the passenger compartment or the garage reaches a concentration that affects the human body, whereby the concentration of the noxious component can be prevented from being more than or equal to the concentration that affects the human body.

Although the present disclosure has been described and illustrated in detail, it is clearly understood that the same is by way of illustration and example only and is not to be taken by way of limitation, the scope of the present disclosure being interpreted by the terms of the appended claims.

What is claimed is:

1. A vehicle remote control system including a vehicle, the vehicle remote control system comprising:
a control unit that starts a change of a state of the vehicle to a predetermined state when a predetermined start condition is satisfied;
a calculation unit that calculates an estimated required period required to change to reach the predetermined state; and
a notification unit that notifies, to a user, estimation information about the estimated required period calculated by the calculation unit.

2. The vehicle remote control system according to claim 1, wherein
the predetermined start condition is a condition that a remote instruction is received from the user, and
the estimation information includes an estimated time at which the state of the vehicle reaches the predetermined state.

3. The vehicle remote control system according to claim 1, wherein
the predetermined start condition is a condition that a time to start the change of the state of the vehicle to the predetermined state is reached, the time to start the change of the state of the vehicle preceding, by the estimated required period, a scheduled departure time set previously, and
the estimation information includes a time at which the predetermined start condition is satisfied.

4. The vehicle remote control system according to claim 1, wherein
the vehicle includes
a window, and
a removal device that removes, by heat, a deposit originating from water and adhered to the window,
the predetermined state is a state in which the deposit is removed by the removal device to such a predetermined criterion that a field of view of the user through the window during driving of the vehicle is able to be secured, and
the control unit controls the removal device to start the change of the state of the vehicle to the predetermined state.

5. The vehicle remote control system according to claim 1, wherein
the vehicle includes an air conditioner in a passenger compartment,
the predetermined state is a state in which inside of the passenger compartment is air-conditioned to a predetermined temperature by the air conditioner, and
the control unit controls the air conditioner to start the change of the state of the vehicle to the predetermined state.

6. The vehicle remote control system according to claim 1, further comprising a collection unit that collects information, used to calculate the estimated required period, about the state of the vehicle, wherein
the calculation unit calculates the estimated required period using the information collected by the collection unit.

7. The vehicle remote control system according to claim 6, wherein the information about the state of the vehicle is weather information used to calculate the estimated required period.

8. The vehicle remote control system according to claim 6, wherein the information about the state of the vehicle is information, used to calculate the estimated required period, about a period of time from start of a change of a state of another vehicle to start of driving of the another vehicle.

9. The vehicle remote control system according to claim 1, wherein
the vehicle includes
an internal combustion engine, and
a sensor that detects a concentration of a noxious component in an external air, the noxious component being included in exhaust gas from the internal combustion engine,
the control unit starts to change the state of the vehicle to the predetermined state by operating the internal combustion engine, and
when the concentration detected by the sensor is more than or equal to a predetermined value, the control unit stops the internal combustion engine.

10. A vehicle remote control method in a vehicle remote control system including a vehicle, the vehicle remote control method comprising:
starting a change of a state of the vehicle to a predetermined state when a predetermined start condition is satisfied;
calculating an estimated required period required to change to reach the predetermined state; and
notifying, to a user, estimation information about the calculated estimated required period.
